(19) United States
(12) Patent Application Publication
(10) Pub. No.: US 2008/0188928 A1
(43) Pub. Date: Aug. 7, 2008

(54) Medical device delivery sheath

(76) Inventors: Amr Salahieh, Saratoga, CA (US); Emma Leung, Sunnyvale, CA (US); Daniel Hildebrand, Menlo Park, CA (US); Jonah Lepak, Santa Cruz, CA (US); Ulrich R. Haug, Campbell, CA (US); Dwight Morejohn, Davis, CA (US); Tom Saul, El Granada, CA (US)

Correspondence Address: SHAY GLENN LLP, 2755 CAMPUS DRIVE, SUITE 210, SAN MATEO, CA, 94403, US

(21) Appl. No.: 11/314,183

(22) Filed: Dec. 20, 2005

Related U.S. Application Data
(60) Provisional application No. 60/717,916, filed on Sep. 16, 2005.

(51) Int. Cl. A61F 2/24

(52) U.S. Cl. 623/2.11

(57) ABSTRACT

A support member for a catheter sheath is disclosed. The support member has a series of ribs with a distal member having integrated fingers for providing radial compliance. The support member provides sufficient axial stiffness to provide a desired pushability of a minimally invasive device for replacing a heart valve. Various alternative embodiments are also described.

### CROSS-REFERENCE

This application claims the benefit of U.S. Provisional Application No. 60/717,916; filed Sep. 16, 2005, which is incorporated herein by reference in its entirety.

### BACKGROUND OF THE INVENTION

The present invention relates to sheaths for use with catheters or other endovascularly or laparoscopically delivered devices.

One of the most important advances in surgery over the last few decades has been the adoption and routine performance of a variety of minimally invasive procedures. Examples of minimally invasive procedures include angioplasty, endoscopy, laparoscopy, arthroscopy and the like. Minimally invasive procedures such as these can be distinguished from conventional open surgical procedures in that access to a site of concern within a patient is achieved through a relatively small incision, into which a tubular device (such as a catheter) is inserted or introduced. The tubular device or device portion keeps the incision open while permitting access to the surgical site via the interior lumen of the tube. The tubular device also provides a pathway for delivery of tools and implanted devices to a target site within the patient.

Inherent in the performance of minimally invasive procedures is the need to provide surgical tools and implantable devices that can be introduced into the patient through these access lumens. These tools and devices may need to be advanced through bent and narrow body passages (such as blood vessels); the tools and devices must therefore be sufficiently flexible to negotiate bends and turns while sufficiently stiff to enable their distal ends to be moved in response to movement of their proximal ends by a user. The diameter of the access lumen limits the diameter of such tools and implants which, among other effects, may limit the stiffness and/or flexibility of the tools and implants.

For example, endovascularly delivered medical devices are often delivered through a patient's vasculature via a catheter or sheath. Examples of such a device and delivery system are disclosed in U.S. Patent Appl. Publ. No. 2005/0137688 and U.S. Patent Appl. Publ. No. 2005/0137699 which describe replacement heart valves and aspects of their delivery systems. One aspect of these systems is the use of a sheath in the deployment of the implant. The delivery sheath must be stiff enough to advance the device through the vasculature, but compliant enough to negotiate the sometimes tortuous turns of the patient's vasculature. In addition, the sheath may need to be steered during advancement through the vasculature.

Also, it may be desirable to bring an expandable device back into the sheath after initial deployment of the device from the sheath for removal from the patient or possible redeployment within the patient. If the device is to be redeployed or otherwise reused, or if damage to the device is otherwise undesirable, this resheathing must be done without harming the device. In addition, devices delivered via the sheath must first be loaded into the sheath. The sheath might therefore be required to help collapse the expandable device in a non-harmful manner during the initial loading or following resheathing operations. This activity could be difficult in situations in which a relatively high force is required to collapse the expanded device. Thus of particular interest in the present discussion is the development and construction of sheath like tubes to assist in the deployment of an implantable device.

### SUMMARY OF THE INVENTION

The present invention provides a sheath having the advantages of the prior art while including the added features of controlled bending combined with radially expandability of the tip to facilitate the collapse and resheathing of a medical device.

The invention also provides catheter or sheath having a distal tip segment requiring a lower force to expand the distal tip, thus reducing the stress and axial compression forces associated with sheathing or resheathing an implant.

The invention provides a medical device delivery sheath having one or more of the following features: Sufficient axial stiffness to enable advancement of the sheath through the vasculature; sufficient bending compliance to permit movement of the sheath through bends in the vasculature; sufficient axial stiffness to enable the application of a sheathing force to collapse an expandable device into the distal end of the sheath; and distal end features that accomplish sheathing of the expandable device without harm to the device or delivery tool.

One aspect of the invention provides a catheter with a radially expandable tip. The tip has a cuff with a first end and a second end distal to the first end and a polymer jacket surrounding the cuff. The second end of the cuff has a plurality of irregular tabs. The cuff and the polymer jacket are adapted to allow the second end of the cuff to expand more easily than the first end of the cuff in response to an axially directed force on the tip of the catheter.

In another aspect of the invention, there is a delivery tool for endovascularly delivering a replacement heart valve. The delivery tool has a sheath for assisting in the deployment of the replacement heart valve. The sheath has a support member having a rib cage structure with at least one spine and a plurality of ribs. There is also a polymer jacket surrounding the support member.

In yet another aspect of the invention there is a support member for a catheter, the support member having a spine and a substantially continuous rib cage substantially along the length of the spine.

Still another aspect of the invention provides an endovascular valve delivery system having a deployment tool and an implant. The deployment tool has a proximal end, a distal end and a sheath. The sheath has at least a proximal zone and a distal zone, wherein the distal zone of the sheath has a reduced radial stiffness from the proximal zone such that the distal zone may be expanded to form a funnel. The implant is releasably engaged to an aspect of the deployment tool distal end and is adapted to be drawn into the sheath by engaging the implant with the sheath to expand the sheath distal zone into a funnel.

Yet another aspect of the invention provides a system for endovascular replacement of a heart valve. The system has a handle, a deployment tool attached to the handle, a replacement heart valve and a sheath. The deployment tool has one or more actuation elements extending therethrough. The replacement heart valve is releasably engaged to the deployment tool. The sheath extends substantially over the length of the deployment tool and replacement heart valve and has a support member with a first zone of uniform stiffness, the first zone extending substantially over the entire length of the deployment tool; and a second zone of variable stiffness, the second zone forming a distal tip of the sheath, and capable of expansion to form a funnel for assisting in the capture of the replacement heart valve.

Still another aspect of the invention provides a method of drawing a replacement heart valve into a deployment system having an inner member deployment tool member in sliding engagement with an outer sheath member, the deployment tool member supporting the replacement valve, the sheath member having a radially expandable tip. The method includes the steps of holding one of the members in a substantially stationary position relative to the patient, and moving the other member relative to the stationary member to draw the replacement heart valve within the sheath member.

Yet another aspect of the invention provides a method of deploying a replacement heart valve using a deployment system having an inner deployment tool member and an outer sheath member, wherein the deployment tool member and the sheath member are releasably engaged to an implant. The method includes the steps of moving at least one of the members with respect to the other member to bend a tip of the sheath member and releasing an engagement between the sheath member and the implant.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1A illustrates a system for deploying a replacement heart valve.

FIGS. 1B-4 provide basic illustrations of a sheath with various support members.

FIG. 5 illustrates a rib cage support structure.

FIGS. 6-7 show cross sections of a sheath.

FIGS. 8-11 illustrate the flexibility of the support member.

FIG. 12 shows rib cage elements in compression during bending.

FIGS. 13-34 provide various examples of support member patterns.

FIGS. 35-36 show two possible end sections of the support member.

FIG. 37A-B illustrate the sheath in operation.

FIGS. 38-39 provide a cross section view of a sheath with an expanding tip.

FIGS. 40A-46B provide patterns of the irregular tabs in the tip.

FIGS. 47-49 show various wire support designs.

FIGS. 50-51 illustrate nose cone variations.

FIGS. 52-55 illustrate an implant being received by the sheath tip.

FIGS. 56-63 illustrate purse string type embodiments of the sheath closure device.

FIG. 64 shows a nosecone support employing aspects of the invention.

FIGS. 65-67 show alternative patterns for the nosecone support of FIG. 64.

### DETAILED DESCRIPTION OF THE EMBODIMENTS

The present invention provides for a delivery sheath for use as part of an implant deployment system. In some embodiments, the deployment system is characterized by having numerous actuating elements for the mechanical operation of various movable parts used to engage and deploy an implant. A specialized sheath is desirable for use as part of the deployment system to provide the ability of deploying and sheathing the implant without harming the implant or deployment tool. Furthermore, the sheath described herein has sufficient axial stiffness to enable advancement of the sheath through the vasculature while retaining sufficient compliance to permit movement of the sheath through bends in the vasculature. In addition, the sheath incorporates a distal tip having sufficient stiffness to enable the application of a sheathing force to collapse an expandable device so that it may be drawn into the distal end of the sheath. It is desirable to minimize sheathing force in order to facilitate delivery and sheathing of the implant as well as reduction of delivery tool deformations during sheathing. Desirably neither the implant nor the sheath are harmed, damaged or plastically deformed during the intended use of the deployment system having a sheath as described herein. In some embodiments, the sheath has a deformable tip that provides a lead-in for sheathing an implant, provides protection against tip inversion during implant sheathing, and/or can mimic a catheter nosecone. In some embodiments the sheath has a radially expandable tip that provides improved distal fitting over a fixed nosecone or guidewire. Because a sheath with expandable distal tip has greater flexibility and can be manufactured in a variety of specialized configurations, it can provide increased reliability and/or smaller profiles.

One embodiment provides a catheter with a radially expandable tip. The tip comprises a cuff and a polymer jacket. The cuff has a first (proximal) end and a second (distal) end. The second end has a plurality of irregular tabs extending from it. A tubular polymer jacket surrounds the cuff, and the cuff is mated to the catheter at the first (proximal) end, such that the irregular tabs on the second (distal) end allow the tip to expand with a webbing of polymer material between the tabs. In an alternate embodiment the polymer jacket conforms more closely to the shape of the tabs such that there is no webbing between tabs.

In this embodiment, the cuff is produced as a feature of the support member that is incorporated in the sheath. In an alternate embodiment the cuff with its polymer jacket may be initially produced as a separate component and then added onto the distal end of a sheathing catheter. The cuff is stiffer than its surrounding polymer jacket. The cuff may be made of metals like stainless steels, nickel-titanium (NiTi) blends or polymers. The proximal end of the cuff can be mated directly to a physical structure in the sheath, or it can be surrounded by a polymer jacket, and then joined to a sheathing catheter. The irregular shaped tabs of the second end provide structural fingers in a substantially axial alignment to the sheath, as well as axially aligned apertures allowing for the tabs to flex apart from each other and provide the tip with radial expandability. It is not necessary or required that the tabs have a tapering or patterned shape so that the radial force needed to expand the tip decreases as one moves distally. The tabs may have a neck down region near the junction with the cuff so there is an intermediate region of very low radial resistance to any expansion force exerted on the tip. This provides a hinge like feature in the tip, particularly when the polymer jacket has a sufficiently high elasticity to conform to different radial diameters.

The shape and material of the tabs, combined with the material and thickness of the jacket should combine to form a tip having a lower radial stiffness than the proximal body of the sheath. The tip composition allows the sheath tip to expand while being advanced over an expanded element of the deployment tool. The expanded portion of the sheath exerts an inward radial force on the expanded element of the deployment tool to assist in the radial collapse of the deployment tool, and thus assist in the collapsing of the implant. Similarly the sheath may expand while the expanded element of the deployment tool is retracted as the sheath is held in a substantially stationary position relative to the patient. The irregularly shaped tabs may be strips of varying length that are axially aligned with the sheath.

In another embodiment, there is a delivery tool for endovascularly delivering a replacement heart valve. The delivery tool has a sheath for assisting in the deployment of the replacement heart valve. The sheath comprising a support member having a rib cage structure with at least one spine and a plurality of ribs. There is also a polymer jacket surrounding the support member. The support member may also have a plurality of axially aligned wires. The wires may extend the entire length of the sheath, or they may be deployed in partial lengths along the sheath and may have areas of over lap. The wires are incorporated between the layers of the polymer jacket so as to avoid any injury to the patient during use of the medical device. The delivery tool in this embodiment may incorporate an extruded body having a plurality of lumens. These lumens act as pathways for a series of actuation elements such as threads or wires. The extruded body is used along with the actuation elements to deploy a replacement heart valve having a mechanically controlled length compression aspect to assist in the deployment of the replacement heart valve.

In yet another embodiment there is a support member for a catheter, the support member comprising a substantially continuous rib cage. In this embodiment, the spine extends substantially the entire length of the sheath. Hoops are attached or incorporated into the spine at intervals along the length of the spine, and the hoops act as ribs for radial structural support. The hoops form structural members to help define the lumen of the sheath, and ensure the lumen does not collapse when the sheath is being used. The hoops may be aligned in a perpendicular fashion to the spine, or they may be at an off angle such that the hoops give the appearance of being in a spiral configuration about the spine. The spine and hoop members may be laser cut from a hypo tube having the desired physical characteristics for the sheath. Characteristics such as having an inner diameter, outer diameter and material thickness suitable for a medical device sheath, along with appropriate mechanical or material properties. The hoops may have a staggered arrangement so that in some lengths along the spine, the hoops are closer together while in other lengths of the spine they may be further apart, and in some lengths the hoops may be absent. The hoops may also be cut so they have a variety of different orientations relative to each other, as well as different profiles.

In another embodiment there is an endovascular valve delivery system comprising a deployment tool and an implant. The deployment tool has a proximal end, a distal end and a sheath. The sheath has a proximal zone and a distal zone, wherein the distal zone of the sheath has a reduced radial stiffness from the proximal zone such that the distal zone may be expanded to form a funnel. The implant is releasably engaged to the deployment tool distal end and adapted to be withdrawn into the sheath where the withdrawing process is facilitated by the funnel.

In yet another embodiment, there is a system for endovascular replacement of a heart valve, the system having a proximal end and a distal end. The system comprising a handle, a deployment tool comprising a sheath, and a replacement heart valve. The handle is proximally located with the deployment tool fixedly attached to the handle. The deployment tool has one or more actuation elements extending there through. The replacement heart valve is distally located and releasably engaged to the deployment tool. The sheath extends substantially over the length of the deployment tool and replacement heart valve, the sheath having a support member comprising a first zone of uniform stiffness, the first zone extending substantially over the entire length of the deployment tool; and a second zone of variable stiffness, the second zone forming a distal tip of the sheath, and capable of expansion to form a funnel for assisting in the capture of the replacement heart valve during deployment.

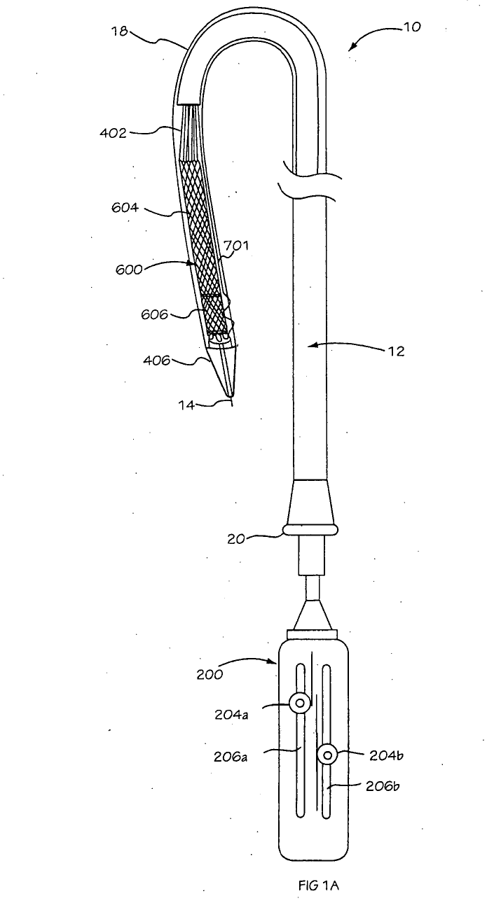

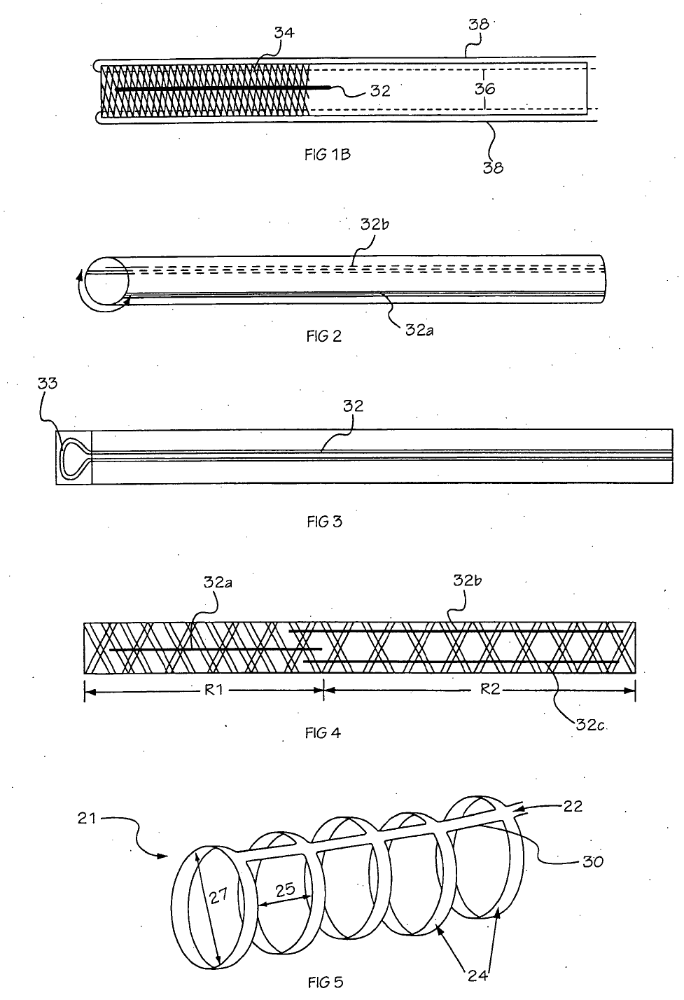

FIG. 1A shows an implant system 10 designed with a deployment tool 12 designed to delivery and deploy an implant 600, such as a replacement heart valve 606 and anchor 604, through a patient's vasculature to the patient's heart. Actuators, such as actuators 204a, 204b, in a handle 200 proximal of the deployment tool 12 provide force and/or displacement to the implant 600 or to other aspects of the deployment tool. As shown, the system 10 also has a guide wire lumen for slidably receiving a guide wire 14, a nose cone 406 for facilitating advancement of the system 10 through the vasculature, an outer sheath 18, and an outer sheath advancement actuator 20. A more thorough description of the system is provided in co-pending U.S. patent application Ser. No. filed Nov. 11, 2005, titled “Medical Implant Deployment Tool.”.

Sheath 18 has a unique combination of capabilities. The sheath is desirably flexible enough to navigate the vasculature, while simultaneously exhibiting sufficient radial compliance on its distal end to allow the sheath to expand and receive the implant 600 (“sheathing”). In this embodiment, advancement of sheath 18 with respect to deployment tool 12 and implant 600 (or retraction of deployment tool 12 and implant 600 with respect to sheath 18) applies a radially inward force upon actuation elements 402 of the deployment tool, which are attached to implant 600. This action draws elements 402 radially inward as the device moves into the sheath. Also, since implant 600 is attached to elements 402, implant 600 also begins to contract radially, with elements 402 providing a mechanical advantage for the radial contraction that reduces the overall force required to be transmitted through the sheath tip during sheathing. When sheath 18 meets implant 600 as the sheathing operation proceeds, any further radially contraction of implant will occur so that implant 600 is fully drawn into sheath 18.

In addition to flexibility and expandability of the distal end, the sheath also desirably possesses sufficient axial stiffness for easy advancement (pushability) through the patient vasculature. FIG. 1B shows one embodiment of a sheath. The sheath has an inner liner 36 and an outer liner 38 such as a polymer jacket. Sandwiched between the inner liner 36 and outer liner 38 are one or more support members having variable axial stiffnesses. For example, a general wire braid 34 can be incorporated for general support. The wire braid is preferably not so stiff, however, as to prohibit the distal end from expanding or (in some embodiments) contracting. If a wire braid is used for structural support in a distal section, it can have different properties from the wire braid used in a proximal section. Alternatively or additionally, axial wires 32 may be woven into the braided wire layer, or may be laid to either the outer surface or inner surface of the braided wire layer. The outer liner can be used to hold the axial wire in place. A single axial wire or stiffener may be used. The stiffener may be a polymer filament having a higher modulus than the polymer jacket material, or the wire may be any of a variety of metal alloys such as stainless steel or Nitinol. Multiple axial elements may be incorporated into the sheath (FIG. 2).

The wire or filament may be formed into a distal loop 33 (FIG. 3) to provide an atraumatic end. Where there are multiple wires 32a, 32b, 32c or filaments, the wires may not be continuous from the proximal end to the distal end (FIG. 4). For example, differing regions of radial compliance are indicated in FIG. 3 as a lower radial compliance R1 region and a higher radial compliance region R2. A balancing of axial stiffness and radial compliance can be achieved by providing for either stiffer axial support members along discrete lengths of the sheath, or a greater number of more compliant axial support elements along the same region. Regions of radial compliance can be achieved by varying the wire braid density. When axial flexibility/bending is required, fewer stiffening elements may be used. Thus the wire wrapping (e.g., density of wrapping turns, thickness of wrapping wire) may differ in different sections of the sheath length, as well as the distribution and/or density of the axial wires in different sheath sections as well.

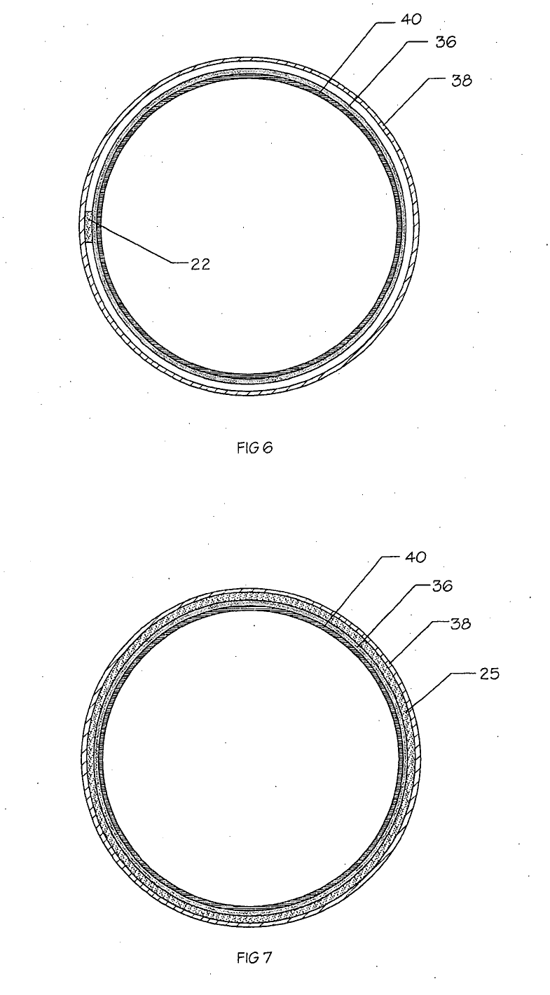

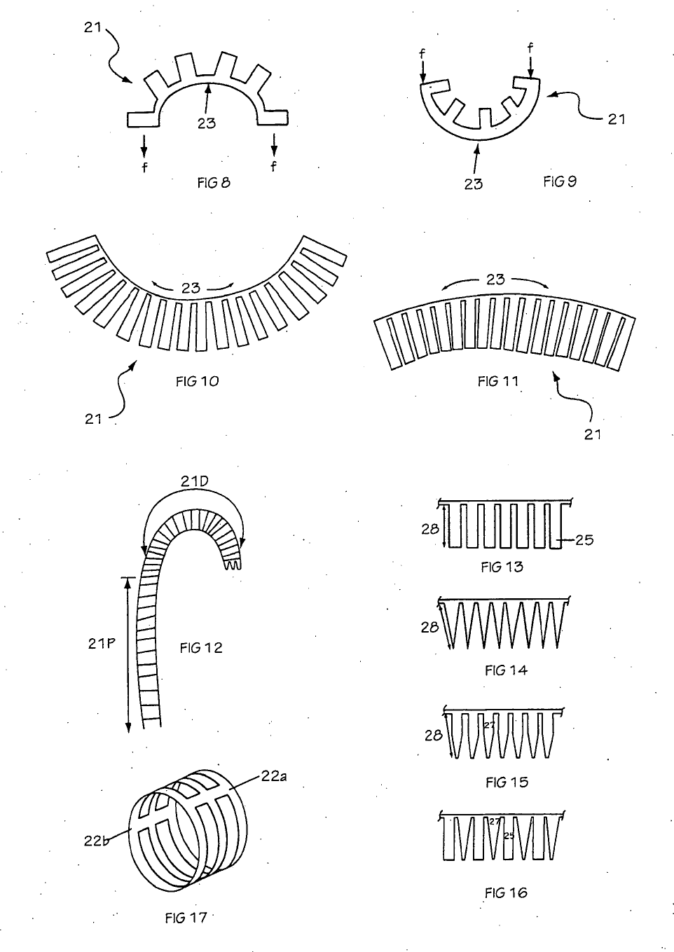

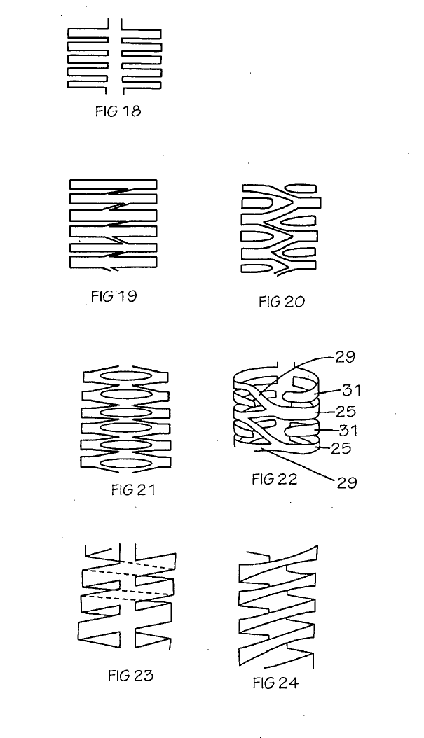

FIG. 5 shows another embodiment of a support member using ribs 24 connected by a spine 22 to support a sheath. The spine 22 provides axial stiffness, while the ribs 24 permit bending, particularly about the narrow cross-sectional dimension of the spine. The ribs provide reinforcement in the form of radial stiffness to the sheath lumen 27 while allowing for great bending flexibility. FIGS. 6 and 7 show cross-sections of a sheath, one at a spine 22 and one at rib 24 disposed within inner liner 36 and outer liner 38. FIG. 6 shows a cross-section taken between two ribs (showing spine 22), while FIG. 7 shows a cross-section taken through a rib 24. A lubricious coating 40 is also shown in the interior of the sheath to reduce friction between the sheath, the implant, and the deployment catheter. Bending compliance of the sheath depends on the direction of the bend as well as the dimensions of the ribs and spine as well as the gap spaces between the ribs, as shown in FIGS. 8-11. Here the support member 21 is shown being flexed both toward the spine side 23 and away from the spine side. If the gap space 25 is large, the support member 21 has a smaller bend radius (FIGS. 8-9). If the gap spaces 25 are narrow, then the support member has a correspondingly larger bend radius (FIGS. 10-11). The support members may also be designed so the rib elements physically interfere with each other (FIG. 11) to ensure the sheath does not bend past a desired minimum radius when deployed.

Rib spacing may be selected so the ribs are close together in regions where bending compliance may be minimized, as along the proximal end of the support member 21P, and made with larger spacing along the distal end of the support member 21D (FIG. 12) where greater bending compliance is required. The ribs may also have a variety of different profiles that enhance or reduce the bending profile along the length of the support member (FIGS. 13-16). As illustrated the differing shape of the ribs allows for a greater amount of flexibility in the bend radius. Ribs having small gap spaces allow less bending compliance as the rib elements will physically interfere with each other as the ribs are bent toward each other. Similarly the ribs will allow greater bending compliance if they are tapered. Rib spacing also allows more room for the liner material to flex and stretch, and can help reduce pinching of the liner material through bending regions. The sheath may be manufactured with a pre-defined shape set, such as a bend which bends away from the spine and away from the gap spaces of the ribs. There may be some regions along the spine with larger gap spaces to promote flexibility while other regions are formed with a wider rib design to promote pushability. Flexibility of the support member will also be affected by the strength and stiffness of the polymer jacket. It is desirable to match the support member to a polymer jacket that will provide for the enhanced features of the support member without canceling out its inherent advantages.

To preserve the gap spaces between the ribs in the bend portions, tapered ribs 27 may be used as shown in FIG. 14-16. Thus over the bend region, the ribs maintain a substantially parallel edge to edge alignment instead of pressing the edges of the ribs together. In this embodiment it may be desirable to use a heat set to provide the sheath with a preferential bend direction so as to promote a favorable position in the human body. This ensures the sheath is bent in the orientation that allows for maximum bending compliance while minimizing stresses on the support member and polymer jacket. Alternating patterns of rib edges may be used (FIG. 16).

The implant system can be steered by using a puller proximate to or diametrically opposed to the sheath's backbone. For example, a steering mechanism releasably coupling the sheath 18 to the implant 600 is shown in FIG. 1A. In this embodiment, the steering mechanism is a wire or thread 701 extending from the proximal handle 200 to the implant 600. At the distal end, wire 701 passes through holes formed in sheath 18 and through holes in the braid of anchor 604. The distal end of wire 701 is releasably attached to the distal end of implant 600, such as by crimping. In use, relative movement between sheath 18 and deployment tool 12 by, e.g., moving handle 200 with respect to sheath actuator 20 (or vice versa) causes the distal tip of implant system 10 to bend in one way or the other. This bend, together with rotation of the entire system within the vascular lumen, can help steer the system as it is advance into the patient's vasculature. When the implant is at the desire site within the patient, an actuator (such as actuator 204a or actuator 204b) in handle 200 can be used to pull wire 701 out of the crimp and through the holes in the anchor and sheath to disconnect the implant from the sheath.

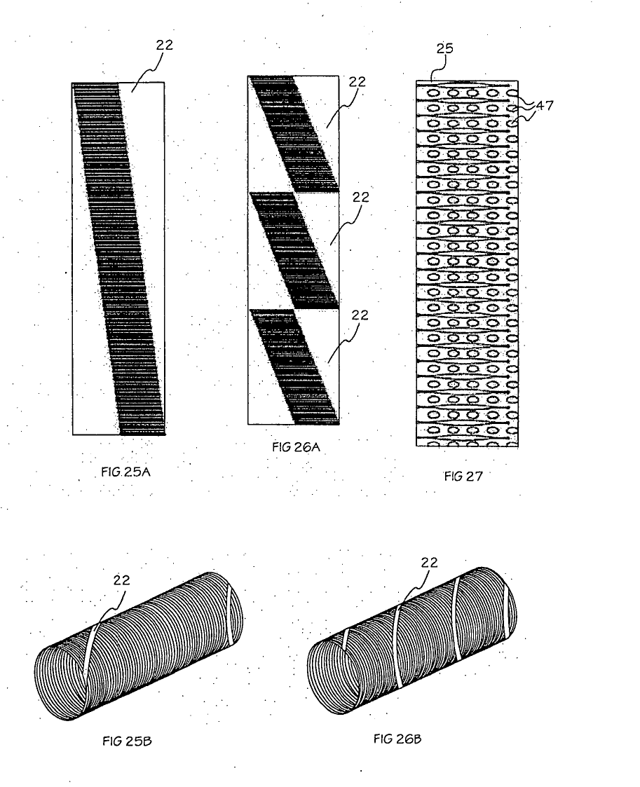

More than one spine may be provided to support the ribs, as shown in FIG. 17, and the spine and ribs may be provided in complex shapes to provide desired bending and axial compliance characteristics along the length of the sheath, as shown in FIGS. 19-24. The support member may also have partial rib segments 31.

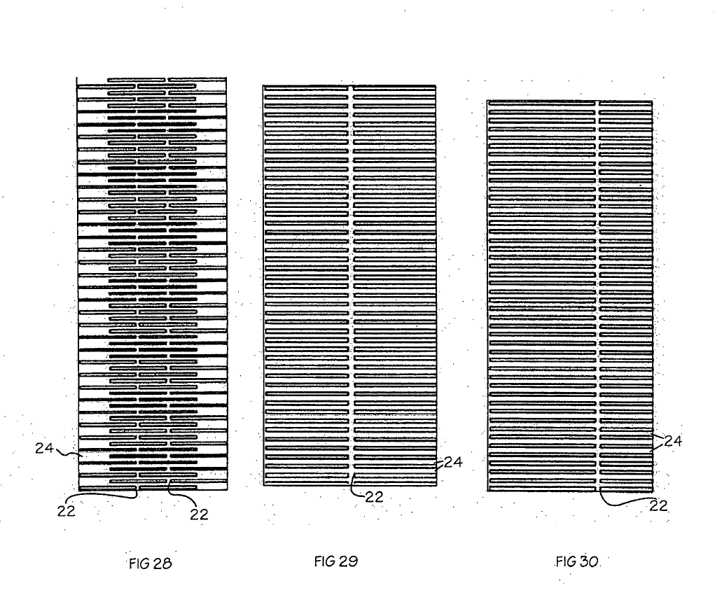

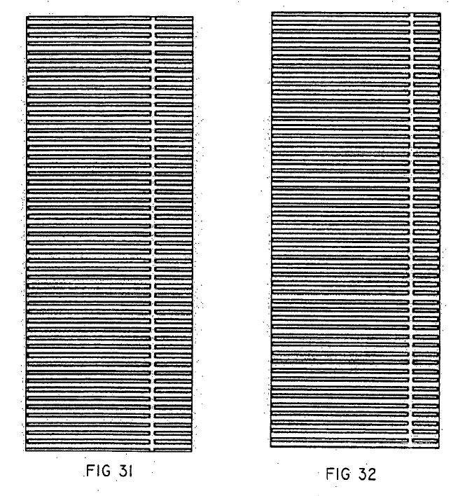

Materials for the ribs and spine may be machined from a high modulus polymer extrusion or laser-cut from a metal tube. FIGS. 25-32 show some of the possible patterns, with the enclosed areas indicating removed material (shown as if the tube had been sliced axially and then flattened out). In FIG. 25, the laser cutting will form a single spine spiraling once around the shown length of the sheath by leaving uncut short lengths between the rectangles as shown in the drawing. One may imagine the spine shifting in an incremental “step wise” fashion, shifting circumferentially around the support member with each gap space. The cut patterns of FIG. 26 yield a single spine wrapping three times around the sheath in the length shown. The cut patterns of FIGS. 27 and 28 yield more complex patterns providing different bending compliance and axial stiffness. FIG. 27 provides a “flat pattern” of the support member having a single spine, and a series of apertures in the rib and spine elements, the apertures similar to those in the design shown in FIG. 36. In FIGS. 29-32, the cut patterns yield two spines arranged 180° apart, 120° apart, 90° apart, and 60° apart, respectively.

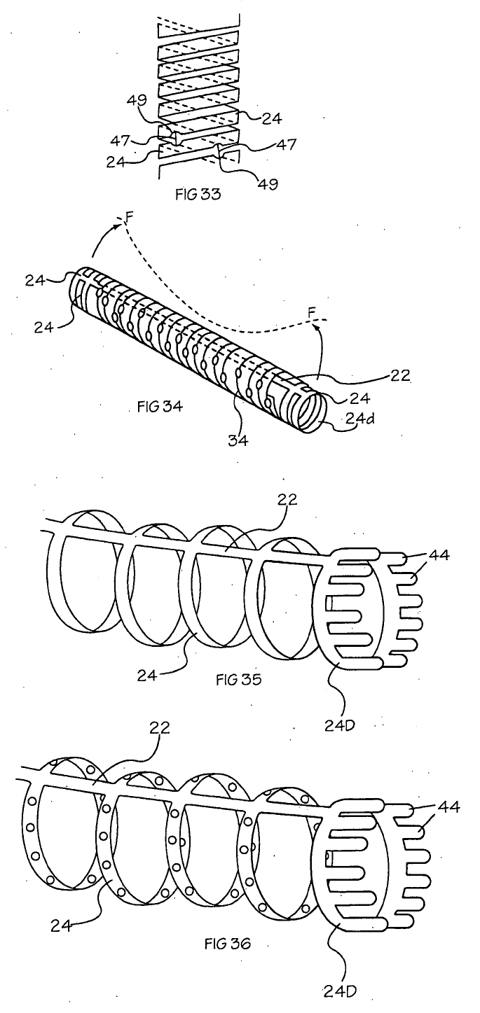

The support member may have additional widened apertures 47 formed among the ribs to provide greater area for the inner and outer liner material to bond between the ribs. The polymer jacket surrounding the support member may be formed of an inner and outer liner having diameters substantially similar to the support member. The inner liner has an outer diameter (OD) just under the inner diameter (ID) of the support member rib cage. The outer liner has an ID just greater than the OD of the support member. The two liners are used to sandwich the support member in between, and are then affixed to each other through heat bonding or chemical bonding. The apertures provide for larger contact area between the two liners and provide for a more robust mating of the inner and outer portions of the jacket. The apertures may be formed between the ribs, so the rib edges have “carve outs” 49 (FIG. 33), or the apertures may be formed in the individual ribs (FIG. 36), providing for a plurality of small mating points between the ribs, or the apertures may be along the spine. Alternatively the support member jacket may be incorporated as part of a dip coating or coextrusion process.

Alternatively, more than one support member having a spine and rib cage design may be combined into a single sheath (FIG. 34). The bending compliance along the length of the compound support member depends on the modulus of the individual support members in combination. Different compliance control configurations may be combined to achieve the desired result. For example, a helical support may surround a spine and rib support, as in FIG. 34. The ribs at the proximal and distal ends of the support provide atraumatic ends for the device. Also, the pitch of the winding of a spiral support may vary along the length of the sheath to provide for different bending compliance along the sheath's length. Other features may be built into the sheath support in addition to the compliance control features discussed above. For example, proximal attachment features may be incorporated into the sheath support. Distal tip features such as those discussed below may be incorporated as well.

In order to minimize damage to the sheath and/or implant during sheathing, the sheath may be provided with a mechanism for reducing sheathing forces. For example, the distal end of the sheath may be more compliant, so that it can expand radially into a funnel shape when forced against the deployment tool actuation elements and/or implant. This reduces compression and strain forces imparted to the sheath during a sheathing process. The reduction of strain and compression forces are desirable to reduce kinking of the sheath and plastic deformation of the support member and liner elements. Furthermore the use of a funnel shape reduces the forces necessary to sheath the implant itself, reducing the risk of damage to the implant and deployment tool, and thus reducing the risk to the patient.

In another aspect of the present invention, the sheath may incorporate structural elements to allow for the expansion of the distal tip (FIG. 35A). In this embodiment, a number of fingers 44 or tab elements are arranged in an axial alignment and extending from the most distal rib 24D of the support member. The fingers allow for a desired level of axial stiffness similar to that of the delivery sheath, while minimizing the effects on radial stiffness. The increased radial compliance may be achieved in a variety of ways. The fingers may be made as part of the support member, or attached to the support member as a separate component. In the case where the support member includes distal fingers, they may be cut as part of the manufacturing of the support member itself. Once again, the inner and outer liners can be bounded to each other through the spacing between the distal fingers. Additionally the outer jacket may not form a tubular structure at the fingers but may be formed to conform to the shape of the fingers as shown in FIG. 35B.

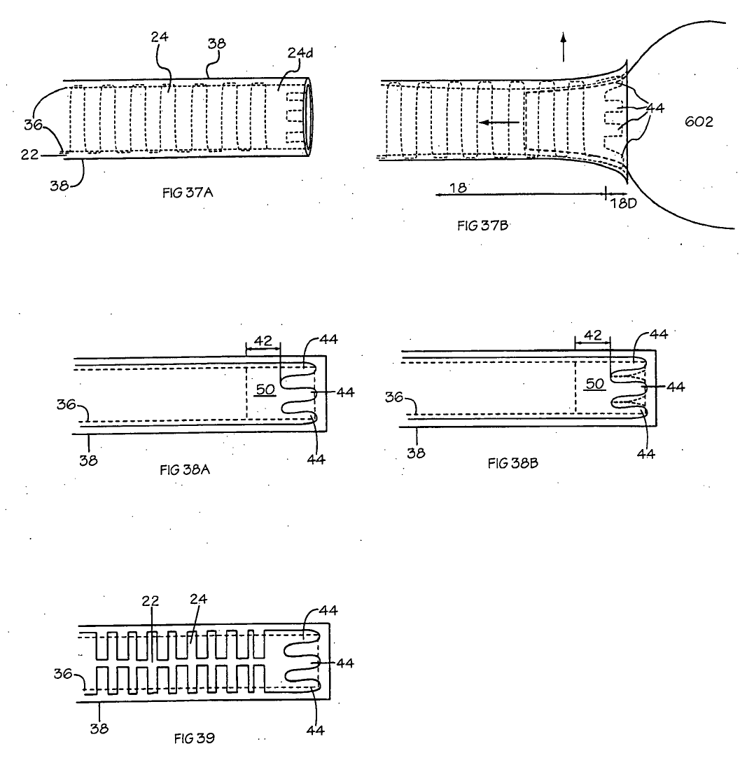

In one operational embodiment, the sheath has a support member sandwiched between an inner liner element and an outer liner element. The support member has a rib like structure along its length running from the proximal end to the distal end. The distal most rib incorporates a plurality of finger-like protrusions that are axially aligned to the sheath. The inner liner and outer liner continue past the tip of the finger protrusions. Thus the liners form a jacket surrounding the entire length of the support member such that no portion of the support member is exposed. The fingers are the only distal elements of the support member (FIG. 37A). As the implant is being drawn into the sheath, the distal end of the sheath expands radially to facilitate the sheathing of the implant (FIG. 37B). The funnel region 18D of the sheath assists in reducing the implant profile during the sheathing operation while simultaneously reducing compression and strain forces on the sheath itself (FIG. 37B).

Other embodiments provide distal end features configured to reduce sheathing forces. For example, the distal end 18D may be configured to have lower radial stiffness than the body of the delivery sheath 18, such as by omitting radial stiffening elements. In other embodiments, the distal tip may use elastomeric materials with a lower durometer than the body of the delivery sheath. As another example, a support within the sheath may have a distal portion with alternating stiffer and more compliant areas, while more proximal portions of the sheath have a more uniform stiffness. This feature is shown schematically in FIGS. 38 and 39, with FIG. 38 representing a sheath that is formed by combining two different sections of stiffener, while FIG. 39 represents an integral stiffener.

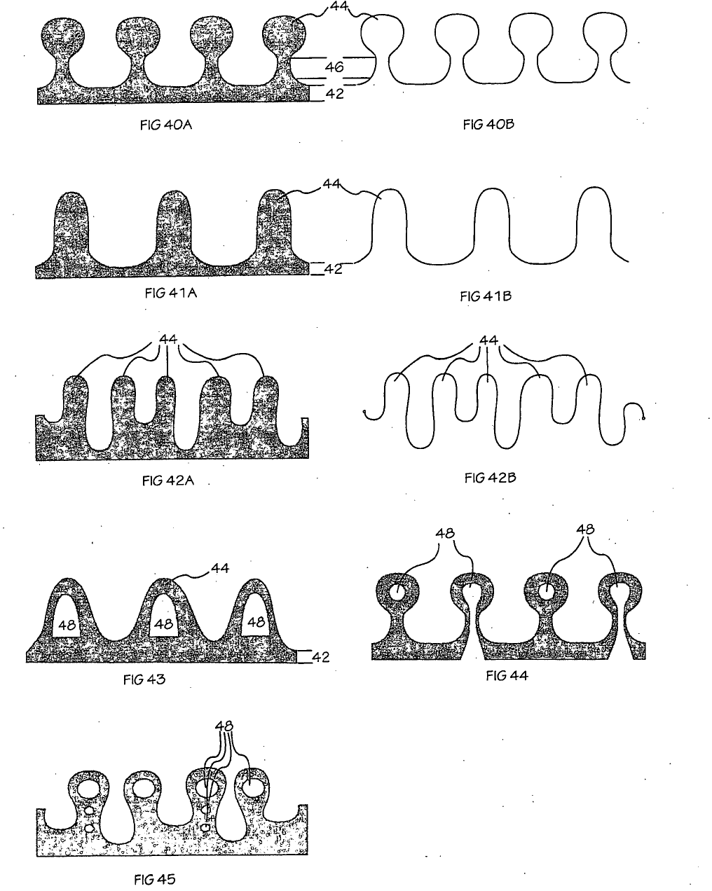

Variations in the design of the support member at the distal tip allow for a wide range of radial expandability and axial stiffness. As shown in FIGS. 40A through 45, the support member may be solid or may be a wire silhouette. The distal end support member incorporating fingers are characterized by a radial stiffness which varies as a function of the distance from the distal cuff to the distal end of the sheath. The finger elements may be fabricated from tubing, flat stock material formed into tubing, injection molded blanks, or wire. The flexural stiffness of the distal tip (or insert) may be decreased at a distance from the tip by having a neck down region (FIG. 40A). Here the fingers are formed as irregularly shaped tabs, having a neck connected to either a cuff, or the distal rib of the support member. The irregular shaped tabs expand and form larger surface area features distal to the neck down region. The neck down region provides enhanced flexibility so the distal end can expand radially, using the neck down region as a sort of hinge, while the larger surface area tabs provide the desired flexural stiffness to funnel the implant and distal deployment mechanism into the sheath. Alternatively the tab elements may be formed from wire with an outline in the same shape as the tabs (FIG. 40B, 41B, 42B)

Alternatively, the tabs may be designed with parallel edges and rounded tips so long as they provide the necessary flexural stiffness and radial compliance (FIG. 42). The irregular shaped tabs may also be formed with one or more apertures within the tab area itself (FIG. 43). This embodiment provides for enhanced mating of the inner and outer liners through the distal end in areas 48. Improved bonding is desirable to prevent the liners from separating or flaying during the deployment and recovery operations for the implant and distal end deployment mechanism. The configuration of FIG. 44 has uneven cutouts for more gradual closing of the sheath. The configuration of FIG. 45 provides a combination of cutout lengths and shapes to provide variable flexural strength and better bonding between sheath layers. These same configurations may be provided using tubular, flat inserts, or wire inserts.

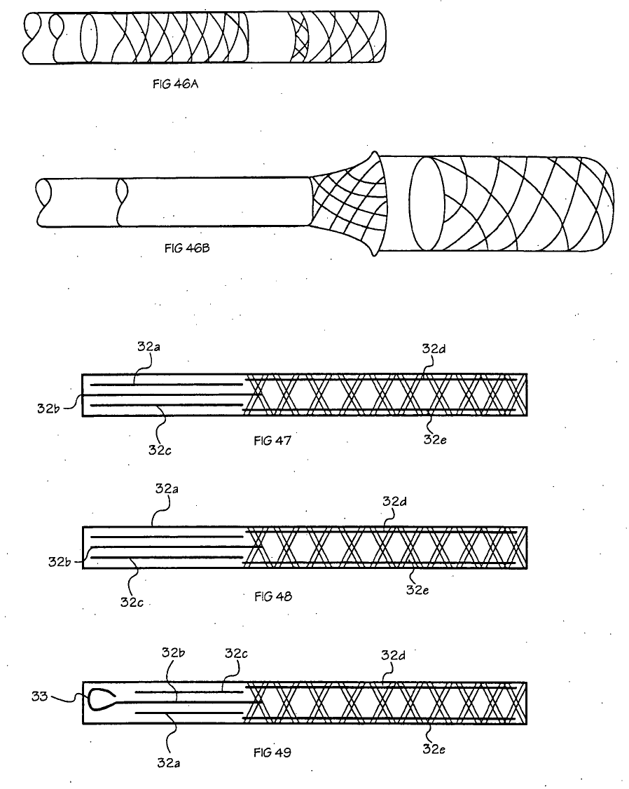

A braided insert may be used to provide the increased radial compliance at the sheath's distal end. In FIG. 46B, the more radially compliant distal end of the braid within the sheath expands to facilitate sheathing. (The implant is shown in an unexpanded configuration within the sheath in FIG. 46A). Alternatively, the braid or helically wound support of a braided sheath may terminate before the distal end of the sheath, as shown in FIGS. 47-49, to permit the distal end of the sheath to be more radially compliant. In FIGS. 47-49, axial wires are embedded in the distal end of the sheath to provide axial compliance. The distal ends of the wires may be staggered, even or looped.

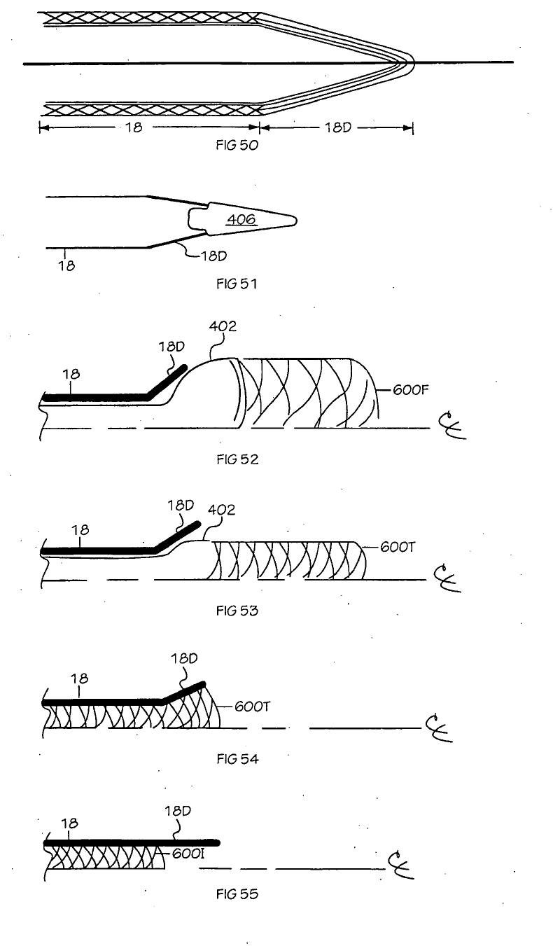

The distal end of the sheath may form a nosecone for the delivery system as shown in FIG. 50. Thus the “at rest” configuration for the delivery sheath distal end may be configured as a continuously decreasing diameter along the distal end. Alternatively the distal end of the sheath may be adapted to close down onto a nose cone 406 (FIG. 51). The nosecone or nosecone interface feature may also be used as a mechanism for reducing resheathing forces on the implant.

In operation, the expandable tip allows for the recapture of the implant once deployed. Initially, the implant is outside the sheath in its enlarged and near final deployed state 600F, while the deployment mechanism is still attached to the proximal end of the implant (FIG. 52). The deployment mechanism has a plurality of actuation elements or fingers 402 that are in physical contact with the ID of the sheath's distal tip 18D. The distal tip is shown expanded so that a funnel is formed. The distal tip of the sheath exerts inward radial force on the actuation elements 402 as the deployment tool is drawn into the sheath (or the sheath is advanced toward the implant) so that the actuation elements contract radially and pull down the implant into a. smaller radial profile. (FIG. 53). As the implant enters the sheath, the funnel of the distal tip of the sheath continues to apply a radially inward force on the implant to reduce the implant's diameter so that it will fit inside the sheath (FIG. 54). Finally the implant is completely sheathed (FIG. 55) and with no force in opposition to the natural radius of the distal tip, the distal tip collapses back into its normal state.

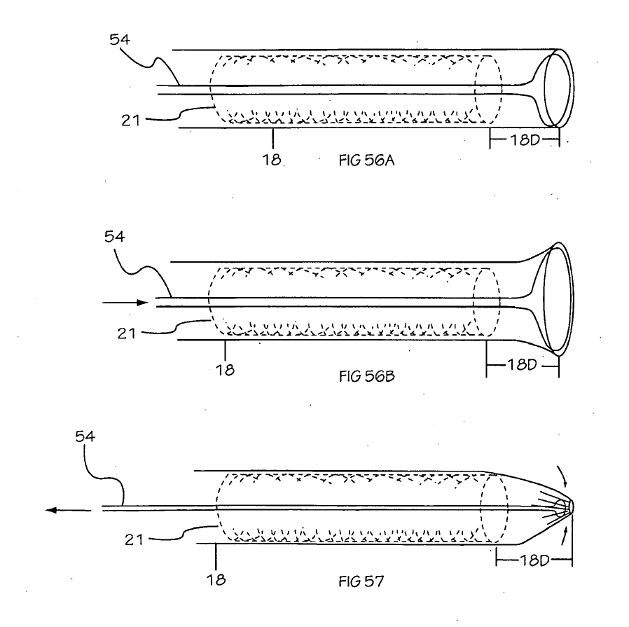

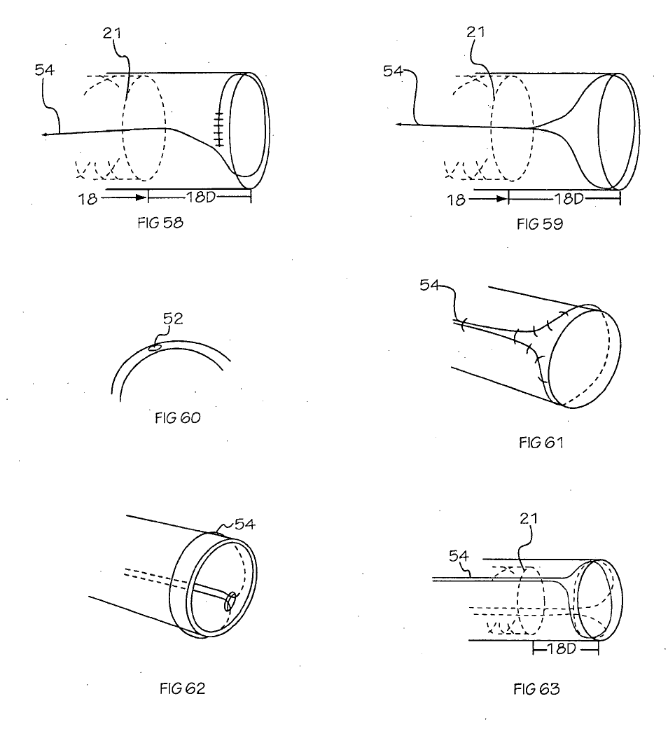

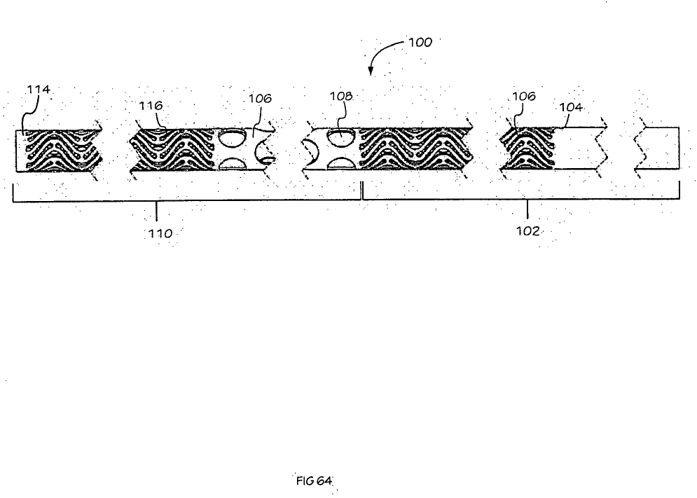

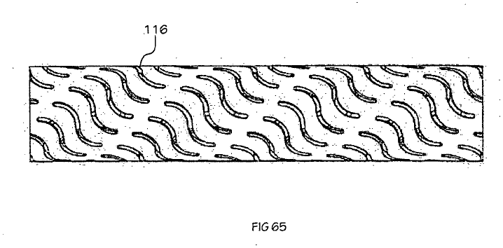

Alternatively, an active mechanical system may be used to control the radial expansion and contraction of the distal tip. A draw string 54, formed from a thread or wire (FIGS. 56A-63) may extend from the proximal end to the distal tip. The draw string 54 may be connected proximally to an actuator in the actuation controller. Distally the draw string forms a loop around the mouth of the sheath. The draw string may be sealed between the inner and outer liner similar to a purse string contained with in a fabric hem. In its neutral position, the draw string allows the distal tip of the sheath to have the same ID as the sheath itself (FIG. 56A). The draw string may be adjusted either manually or automatically. When the implant is being deployed or recaptured, the draw string loosens and allows the distal end to expand (FIG. 56B). Once the implant is captured, or during any period where the deployment tool is navigating the vasculature, the draw string is drawn closed, forming a nose cone at the distal end (FIG. 57).

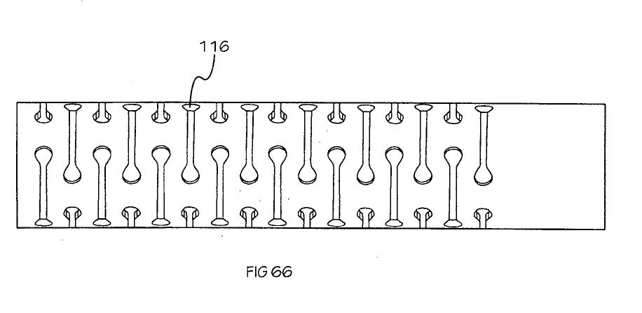

The draw string may have one end affixed to the distal end (FIG. 58) or have both ends extending back to the proximal end of the deployment tool (FIG. 56A, 59). An example of a draw string hem is shown in FIG. 60. The hem is a lumen 52 incorporated into the outer sheath wall. The draw string is desirably tethered at a variety of places both in the distal tip and along the length of the sheath (FIG. 61) to promote the correct and safe operation of the draw string while preventing the material or structure from cinching or collapsing when the draw string is used to reduce the radius of the distal end. Additional draw strings (FIG. 63) may be used to provide control over radial sections of the sheath. Alternatively the draw string may be attached to a slidably movable element of the inner catheter or inner member of the sheath, so that the operation of the draw string does not require a pull down of the draw string along the entire length of the sheath.

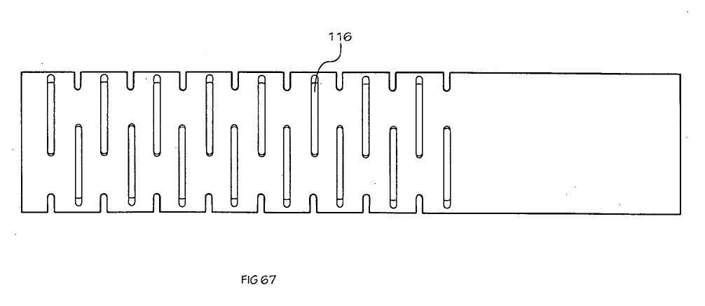

The features providing axial stiffness and bendability to the delivery sheath may also be used in a nosecone support element. FIG. 64 shows a nosecone support element 100 having a nosecone attachment area 102 at its distal end to which a nosecone would be attached. (For purposes of illustration, the nosecone is omitted from FIG. 64.) Element 100 may be made, e.g., from an extrusion or hypotube which is etched or laser cut. Attachment area 102 has two parts, a distal part 104 and a more proximal part 106. Openings 108 are formed in attachment part 106 to provide enhanced gripping areas for the nosecone. Proximal to attachment area 102 is a support area 110 having a more distal part 112 and a more proximal part 114. A series of cut patterns 116 are formed in the distal part 104 of attachment area 102 and in the distal part 112 of support area 110 to enable the nosecone support 100 to bend within the anatomy while still providing axial stiffness and maintaining ultimate axial strength. The more proximal part 114 of support area 110 extends from the implant site back to the device handle (not shown) outside of the patient. In this embodiment, more proximal part does not have any cutouts to facilitate bending, but such cutouts may be provided if desired.

FIGS. 65-67 show alternative cutout patterns for use with nosecone supports. In other embodiments, the nosecone support may not extend proximally to the device handle and is instead supported by other parts of the delivery system.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A catheter comprising a radially expandable tip on a distal end of the catheter, the tip comprising:
a cuff having a first end and a second end distal to the first end, the second end comprising a plurality of irregular tabs; and
a polymer jacket surrounding the cuff,
the cuff and the polymer jacket being adapted to allow the second end of the cuff to expand more easily than the first end of the cuff in response to an axially directed force on the tip of the catheter.

2. The catheter of claim 1, wherein the irregular tabs comprise axially aligned strips of various length.

3. The catheter of claim 1, wherein one or more of the irregular tabs comprises an aperture.

4. The catheter of claim 1, wherein the irregular tabs comprise a plurality of irregular invaginated spaces.

5. The catheter of claim 1, wherein the tip is further adapted to fittingly engage a nose cone.

6. A delivery tool for endovascularly delivering a replacement heart valve, the delivery tool comprising a sheath comprising:
a support member comprising a ribcage structure with at least one spine and a plurality of ribs; and
a polymer jacket surrounding the support member.

7. The delivery tool of claim 6, wherein the support member has a plurality of axially aligned wires.

8. A support member for a catheter, the support member comprising a spine and a substantially continuous rib cage substantially along the length of said spine.

9. The support member of claim 8, wherein the catheter comprises a sheath for an implant deployment tool.

10. An endovascular valve delivery system comprising:
a deployment tool having a proximal end, a distal end, and a sheath, the sheath having a proximal zone and a distal zone wherein the distal zone of the sheath has a reduced radial stiffness compared to said proximal zone such that the distal zone may be expanded to form a funnel in response to an axially directed force; and
an implant releasably engaged to the deployment tool distal end and adapted to be drawn into the sheath by engaging the implant with the sheath to expand the sheath distal zone into a funnel.

11. The system of claim 10, wherein the axial stiffnesses of the proximal zone and the distal zone are different.

12. The system of claim 10, wherein the sheath further comprises an intermediate zone having a different axial stiffness compared to axial stiffnesses of the proximal zone and the distal zone.

14. The system of claim 10, wherein the sheath further comprises an intermediate zone with a different radial stiffness compared to the radial stiffnesses of the proximal zone and the distal zone.

15. The system of claim 10, wherein the proximal zone and distal zone further comprise polymer jackets having different compliance values.

16. The system of claim 10, wherein the distal zone has a variable radial stiffness along its axial length.

17. The system of claim 10, wherein the sheath further comprises:
a support member comprising a ribcage structure with at least one spine and a plurality of ribs; and
a polymer jacket surrounding the support member.

18. The system of claim 17, wherein the plurality of ribs further comprises one or more cutouts.

19. The system of claim 17, wherein the plurality of ribs further comprises one or more apertures.

20. The system of claim 10, wherein the deployment tool is adapted to be steerable.

21. The system of claim 10, wherein the sheath comprises a plurality of substantially axially aligned wires arranged to form a plurality of loops at the distal zone.

22. A system for endovascular replacement of a heart, the system comprising:
a handle;
a deployment tool attached to said handle, the deployment tool comprising one or more actuation elements extending therethrough;
a replacement heart valve releasably engaged to the deployment tool; and
a sheath extending substantially over the length of the deployment tool and replacement heart valve, the sheath comprising a support member comprising:
a first zone of uniform stiffness, said first zone extending substantially over the entire length of the deployment tool; and
a second zone of variable stiffness, said second zone forming a distal tip of the sheath, the second zone being capable of expansion to form a funnel for assisting in the capture of the replacement heart valve.

23. The system as described in claim 22, wherein the support member further comprises at least one intermediate zone disposed between the first zone and the second zone and having a stiffness less than that of said first zone and said second zone.

24. A method of drawing a replacement heart valve into a deployment system having an inner deployment tool member in sliding engagement with an outer sheath member, the deployment tool member supporting the replacement valve, the sheath member having a radially expandable distal tip, the method comprising the steps of:
holding one of the members in a substantially stationary position relative to the patient; and
moving the other member relative to the stationary member to draw the replacement heart valve within the sheath member.

25. The method of claim 24, wherein the moving step comprises the step of operating an actuator mechanically engaged to the moving member.

26. The method of claim 24, further comprising:
repositioning the deployment system;
withdrawing the sheath member with respect to the deployment tool member; and
securing the replacement heart valve into a desired location by actuating a locking mechanism at least partially incorporated into the replacement heart valve.

28. The method of claim 26, further comprising:
separating the replacement heart valve from the deployment tool member.

29. A method of deploying a replacement heart valve using a deployment system having an inner deployment tool member and an outer sheath member, wherein the deployment tool member and the sheath member are releasably engaged to an implant, the method comprising the steps of:
moving at least one of the members with respect to the other member to bend a tip of the sheath member; and
releasing an engagement between the sheath member and the implant.

30. The method of claim 29 wherein the deployment system further comprises an actuator handle, the releasing step comprising moving an actuator on the actuator handle.
